(19) United States
(12) Patent Application Publication
(10) Pub. No.: US 2024/0130657 A1
(43) Pub. Date: Apr. 25, 2024

(54) PHYSIOLOGICAL SENSING DEVICE

(71) Applicant: Industrial Technology Research Institute, Hsinchu (TW)

(72) Inventors: Hsien-Wei Chiu, Taoyuan City (TW); Tai-Jui Wang, Kaohsiung City (TW); Chieh-Wei Feng, Taoyuan City (TW); Jui-Wen Yang, New Taipei City (TW)

(73) Assignee: Industrial Technology Research Institute, Hsinchu (TW)

(21) Appl. No.: 18/454,074

(22) Filed: Aug. 23, 2023

Related U.S. Application Data
(63) Continuation-in-part of application No. 18/155,042, filed on Jan. 16, 2023, now abandoned.

(30) Foreign Application Priority Data
Oct. 17, 2022 (TW) 111139315

(51) Int. Cl. A61B 5/27; A61B 5/00

(52) U.S. Cl. CPC A61B 5/27; A61B 5/6804

(57) ABSTRACT

A physiological sensing device for sensing physiological signal of an organism is provided. The physiological sensing device includes a sensing chip, a coupling sensing electrode and a coupling dielectric stacked layer. The coupling sensing electrode is electrically connected to the sensing chip. The coupling dielectric stacked layer covers the coupling sensing electrode. The coupling dielectric stacked layer is located between the coupling sensing electrode and the organism. The coupling dielectric stacked layer includes a first dielectric layer and a second dielectric layer. The dielectric constant of the second dielectric layer is greater than that of the first dielectric layer. The second dielectric layer is located between the first dielectric layer and the organism.

### CROSS-REFERENCE TO RELATED APPLICATION

This application is a continuation-in-part application of and claims the priority benefit of U.S. application Ser. No. 18/155,042, filed on Jan. 16, 2023, which claims the priority benefit of Taiwan application serial no. 111139315, filed on Oct. 17, 2022. The entirety of each of the above-mentioned patent applications is hereby incorporated by reference herein and made a part of this specification.

### TECHNICAL FIELD

The disclosure relates to a sensing device, and also relates to a physiological sensing device.

### BACKGROUND

In terms of wearable biomedical sensing technology, a physiological signal sensing device (e.g., a sensing electrode patch or a sensor) may be worn on the subject, and various information of the wearer may be recorded at any time in a non-invasive manner, thereby the body temperature, pulse, heartbeat, respiratory rate, and other physiological states of the wearer may be known. In addition, wearable technology may also remind or prevent possible physiological changes, and may even provide prompt and timely reminder and help when symptoms occur. Therefore, wearable biomedical sensing technology is an extremely convenient technological advancement for medical care (e.g., wearer such as patients recuperating at home, patients with a history of heart disease, or the elderly living alone), and may also be used for real-time monitoring of physiological state during exercise. In addition to the type of protective gear or fabric that may be worn on the subject, wearable biomedical sensing devices may be extended to vehicle-mounted devices (e.g., seats or seat belts) and long-term care devices (e.g., wheelchairs or mattresses).

However, based on the limitations of the existing technology, the sensing electrode patch needs to be tightly attached to the skin of the wearer, and prolonged wearing may cause the wearer to experience stress, discomfort, or allergic conditions. Based on this point, the new coupling physiological signal sensing device may reduce the sense of pressure when wearing, but there are problems of weak coupling physiological signal, noise interference/product durability, variation in the material and thickness of the spacer, and sweat interference with the signal. In more detail, although the coupling physiological sensing method adopted may solve the discomfort caused by the conventional impedance sensing, the coupling physiological signal decreases due to the widening of the gap between the sensing electrode and the skin in different usage scenarios (low pressure, non-contact), or the signal is distorted through the transmission line, which affects the interpretation of the physiological signal.

Based on the above, how to increase the coupling capacitance, durability, and reliability of the coupling physiological signal sensing device becomes increasingly important.

### SUMMARY

A physiological sensing device that may effectively increase the coupling capacitance, while taking into account the durability and reliability, is provided in an embodiment of the disclosure.

The physiological sensing device of an embodiment of the disclosure is suitable for sensing physiological signal of an organism. The physiological sensing device includes a sensing chip, a coupling sensing electrode, and a coupling dielectric stacked layer. The coupling sensing electrode is electrically connected to the sensing chip. The coupling dielectric stacked layer covers the coupling sensing electrode and is located between the coupling sensing electrode and the organism. The coupling dielectric stacked layer includes a first dielectric layer and a second dielectric layer, the dielectric constant of the second dielectric layer is greater than the dielectric constant of the first dielectric layer, and the second dielectric layer is located between the first dielectric layer and the organism.

The physiological sensing device of another embodiment of the disclosure is suitable for sensing physiological signals of an organism. The physiological sensing device includes a sensing chip, a coupling sensing electrode, a coupling dielectric stacked layer, and a second dielectric layer. The coupling sensing electrode is electrically connected to the sensing chip. The coupling dielectric stacked layer covers the coupling sensing electrode and is located between the coupling sensing electrode and the organism, the coupling dielectric stacked layer includes a stress compensation layer and a first dielectric layer, and the first dielectric layer is located between the stress compensation layer and the organism. The second dielectric layer is disposed between the stress compensation layer and the coupling sensing electrode.

### BRIEF DESCRIPTION OF THE DRAWING

FIG. 1A and FIG. 1B are cross-sectional schematic diagrams of a physiological sensing device according to a first embodiment of the disclosure.

FIG. 2A to FIG. 2J are cross-sectional schematic diagrams of coupling sensing electrodes and coupling dielectric stacked layers according to various embodiments of the disclosure.

FIG. 3 is a schematic diagram of a pattern of a coupling dielectric stacked layer according to an embodiment of the disclosure.

FIG. 4 is a cross-sectional schematic diagram of a physiological sensing device according to a second embodiment of the disclosure.

FIG. 5 is a cross-sectional schematic diagram of a physiological sensing device according to a third embodiment of the disclosure.

FIG. 6A and FIG. 6B are cross-sectional schematic diagrams of a physiological sensing device according to a fourth embodiment of the disclosure.

FIG. 7A to FIG. 7J are cross-sectional schematic diagrams of coupling sensing electrodes, coupling dielectric stacked layers, and conductive layers according to various embodiments of the disclosure.

FIG. 8 is a cross-sectional schematic diagram of a physiological sensing device according to a fifth embodiment of the disclosure.

FIG. 9 is a cross-sectional schematic diagram of a physiological sensing device according to a sixth embodiment of the disclosure.

### DETAILED DESCRIPTION OF DISCLOSED EMBODIMENTS

The following examples are described in detail with the accompanying drawings, but the provided examples are not intended to limit the scope of the disclosure. In addition, the drawings are for illustrative purposes only and are not drawn in full scale. In order to facilitate understanding, the same elements in the following description are described with the same symbols. In addition, the terms such as “including”, “comprising”, “having”, etc. used in the text are all open-ended terms, that is, “including but not limited to”. Furthermore, wordings used to indicate directions in the disclosure, such as “up,” “down,” “front,” “back,” “left,” and “right,” merely refer to directions in the accompanying drawings, and are not used to limit the disclosure. In addition, the numbers and shapes mentioned in the specification are only used to specifically illustrate the disclosure so as to facilitate understanding of its contents, rather than to limit the disclosure.

An embodiment of the disclosure provides a physiological sensing device including a coupling dielectric stacked layer disposed below the coupling sensing electrode. The coupling dielectric stacked layer may not only increase the coupling capacitance between the sensing electrode and the organism to be measured, but also effectively improve the sensing sensitivity of the physiological sensing device. In the embodiments of the disclosure, the coupling dielectric stacked layer in the physiological sensing device has properties such as scratch resistance, abrasion resistance, and/or moisture resistance, so as to improve the reliability of the physiological sensing device to a certain extent. In addition, the physiological sensing device provided by the embodiments of the disclosure may be compatible with the existing display panel manufacturing process, and has advantages in manufacturing. The physiological sensing device of the disclosure may also be applied in the metaverse, to sense the physiological information of the physical characters, and reflect them on the virtual avatar to increase the interactivity and presence of games or competitive matches.

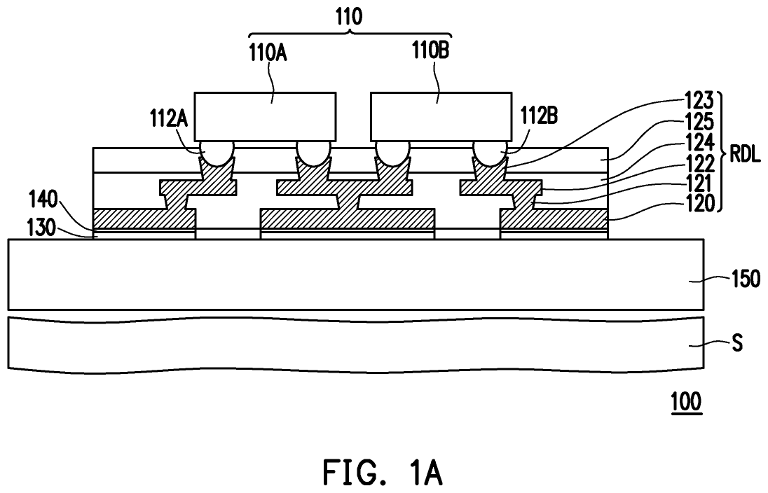

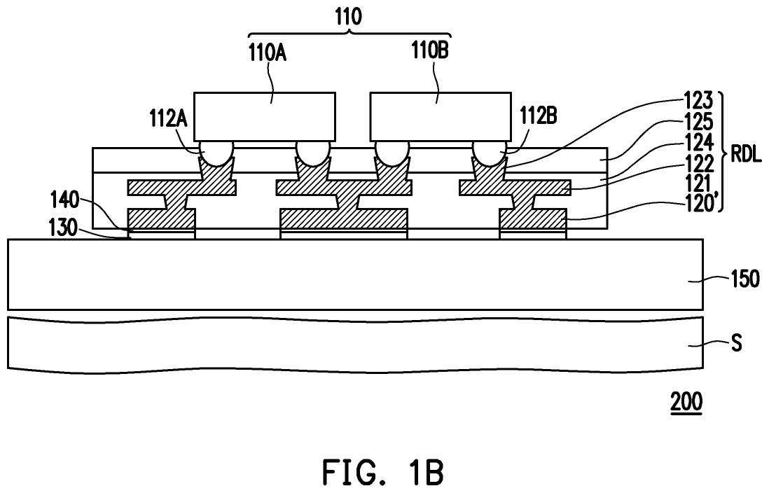

FIG. 1A and FIG. 1B are cross-sectional schematic diagrams of a physiological sensing device according to a first embodiment of the disclosure.

Referring to FIG. 1A, the physiological sensing device 100 of the embodiment is suitable for sensing physiological signal of an organism (e.g., humans or pets), such as electromyography (EMG) signals, electrocardiography (ECG) signals, or electroencephalography (EEG) signals. The physiological sensing device 100 of this embodiment includes at least one sensing chip 110, a coupling sensing electrode 120, and a coupling dielectric stacked layer 130. The coupling sensing electrode 120 is electrically connected to the sensing chip 110, the coupling dielectric stacked layer 130 covers the coupling sensing electrode 120, and the coupling dielectric stacked layer 130 is located between the coupling sensing electrode 120 and the organism (e.g., human skin S). In this embodiment, the sensing chip 110 include a first sensing chip 110A and a second sensing chip 110B. However, the number of the sensing chips 110 is for illustration only, and is not intended to limit the number of the sensing chips 110 used in the disclosure. In some embodiments, the first sensing chip 110A includes a high impedance front end circuit module, and the second sensing chip 110B includes a sensing integrated circuit (IC). As shown in FIG. 1A, the first sensing chip 110A may have a multiple conductive terminals 112A, and the second sensing chip 110B may have multiple conductive terminals 112B, and the conductive terminals 112A and 112B are, for example, solder balls.

In some embodiments, the coupling sensing electrodes 120 may be integrated into a redistribution layer RDL with flexibility, and the redistribution layer RDL may include the coupling sensing electrode 120, a conductive via 121, a noise isolation layer 122, a wire layer 123, multiple dielectric layers 124, and a coupling stress adjustment layer 125. The coupling sensing electrode 120, the conductive via 121, and the noise isolation layer 122 are embedded in the dielectric layers 124, and the coupling sensing electrode 120 is electrically connected to the noise isolation layer 122 through the conductive via 121. In addition, the physiological sensing device 100 may further include a stress compensation layer 140, in which the stress compensation layer 140 is disposed between the coupling sensing electrode 120 and the coupling dielectric stacked layer 130. The stress compensation layer 140 may be formed by a chemical vapor deposition process. By controlling the parameters of the chemical vapor deposition process, the stress compensation layer 140 with a suitable stress value (tensile stress or compressive stress) may be fabricated. The material of the stress compensation layer 140 may be an inorganic material and may include, for example, Pb(ZrTi)O₃, SiO₂, ZnO, Ta₂O₅, Si₃N₄, SiON, BaTiO₃, CaTiO₃, SrTiO₃, TiO₂, MgO, AN, or Al₂O₃.

In this embodiment, the stress value of the coupling dielectric stacked layer 130 may be adjusted in consideration of the overall stress value of the physiological sensing device 100. For example, when the stress value of the physiological sensing device 100 is, for example, 50 Mpa to 200 Mpa (tensile stress), which causes the physiological sensing device 100 to curl, the stress value of the coupling dielectric stacked layer 130 may be adjusted to, for example, −50 Mpa to −200 Mpa to flatten the physiological sensing device 100; when the stress value of the physiological sensing device 100 is, for example, −50 Mpa to −200 Mpa (compressive stress), which causes the physiological sensing device 100 to curl, the stress value of the coupling dielectric stacked layer 130 may be adjusted to, for example, 50 Mpa to 200 Mpa to flatten the physiological sensing device 100.

In this embodiment, the stress compensation layer 140 may improve the stress matching between the coupling dielectric stacked layer 130 and other film layers (e.g., the coupling sensing electrode 120, the noise isolation layer 122, the wire layer 123, etc.) to prevent peeling problems in the manufacturing process. In this embodiment, the stress value of the stress compensation layer 140 may be adjusted in consideration of the stress value of the physiological sensing device 100. For example, when the stress value of the physiological sensing device 100 is, for example, 50 Mpa to 200 Mpa (tensile stress), which causes the physiological sensing device 100 to curl, the stress value of the stress compensation layer 140 may be adjusted to, for example, −50 Mpa to −200 Mpa to flatten the physiological sensing device 100; when the stress value of the physiological sensing device 100 is, for example, −50 Mpa to −200 Mpa (compressive stress), which causes the physiological sensing device 100 to curl, the stress value of the stress compensation layer 140 may be adjusted to, for example, 50 Mpa to 200 Mpa to flatten the physiological sensing device 100. At the same time, the coupling capacitance may still maintain at 1 nF to 10 nF.

It should be noted that the coupling dielectric stacked layer 130 and the stress compensation layer 140 may individually be used to adjust the overall stress of the physiological sensing device 100 or simultaneously be used to adjust the overall stress of the physiological sensing device 100.

The first sensing chip 110A and the second sensing chip 110B may be respectively electrically connected to the redistribution layer RDL below the conductive terminal 112A and the conductive terminal 112B through the conductive terminal 112A and the conductive terminal 112B. Specifically, the first sensing chip 110A may be electrically connected to the coupling sensing electrodes 120 through the conductive terminal 112A, the conductive terminal 112B, the wire layer 123, and the conductive via 121.

The coupling sensing electrode 120 in the redistribution layer RDL is used to sense the physiological signal of the organism. During the sensing of the physiological signal, capacitance is generated between the organism and the coupling sensing electrodes 120, in which the organism is, for example, human skin S. The coupling dielectric stacked layer 130 is disposed below the coupling sensing electrode 120. The noise isolation layer 122 is disposed above the coupling sensing electrode 120, and the coupling sensing electrode 120 and the noise isolation layer 122 overlap each other in the vertical projection direction. In this embodiment, the noise isolation layer 122 may include a ground circuit or a ground patch. The noise isolation layer 122 may prevent the coupling sensing electrodes 120 from directly receiving external noise, resulting in the signal-to-noise ratio (SNR) of the physiological signal being too small to be accurately sensed. The wire layer 123 is disposed above the noise isolation layer 122, and the wire layer 123 is electrically connected to the first sensing chip 110A and the second sensing chip 110B, and the coupling sensing electrodes 120. The wire layer 123 is respectively electrically connected to the first sensing chip 110A and the second sensing chip 110B, for example, through the conductive terminal 112A and the conductive terminal 112B. The layout design of the redistribution layer RDL may be adjusted according to design requirements, which is not limited in the disclosure. The coupling stress adjustment layer 125 is disposed above the dielectric layer 124, and the coupling stress adjustment layer 125 may be used to improve the overall stress matching of the physiological sensing device 100. In some embodiments of the disclosure, the physiological sensing device 100 may further include an insulating encapsulation layer (not shown). The insulating encapsulation layer is disposed above the coupling stress adjusting layer 125 and covers the first sensing chip 110A and the second sensing chip 110B. The insulating encapsulation layer may be filled in the gap between the coupling stress adjustment layer 125 and the first sensing chip 110A and the gap between the coupling stress adjustment layer 125 and the second sensing chip 110B to further laterally cover the conductive terminal 112A and the conductive terminal 112B. In addition, the material of the insulating encapsulation layer is, for example, a soft material, which may include polydimethylsiloxane (PDMS) or thermoplastic polyurethane (TPU), which is used to provide protection and insulation for the first sensing chip 110A and the second sensing chip 110B.

In this embodiment, the material of the coupling sensing electrode 120 may include Mo, Ti, Al, or Cu, and the thickness of the coupling sensing electrode 120 is, for example, 300 nm to 5000 nm.

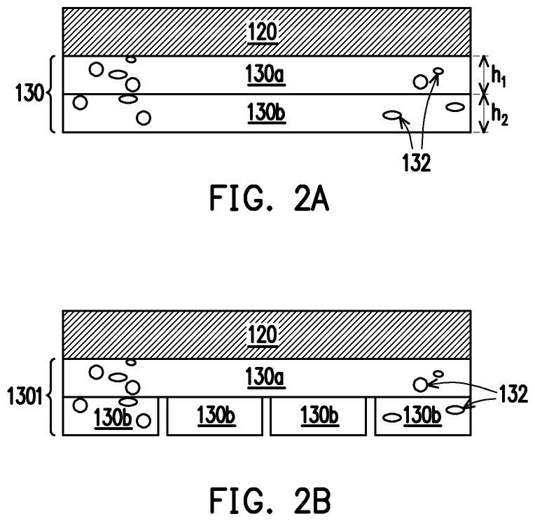

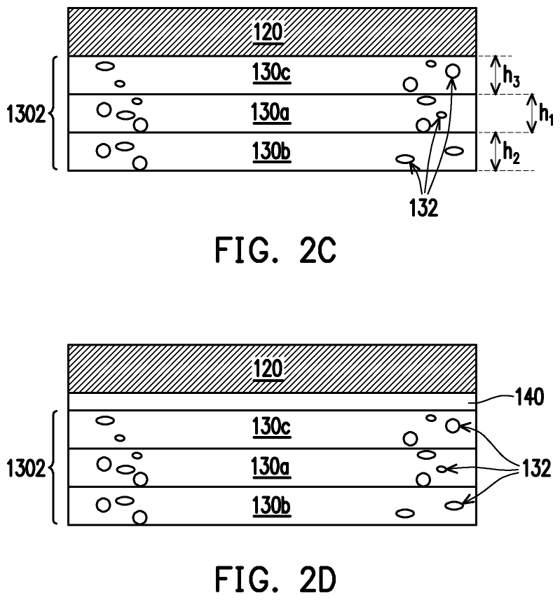

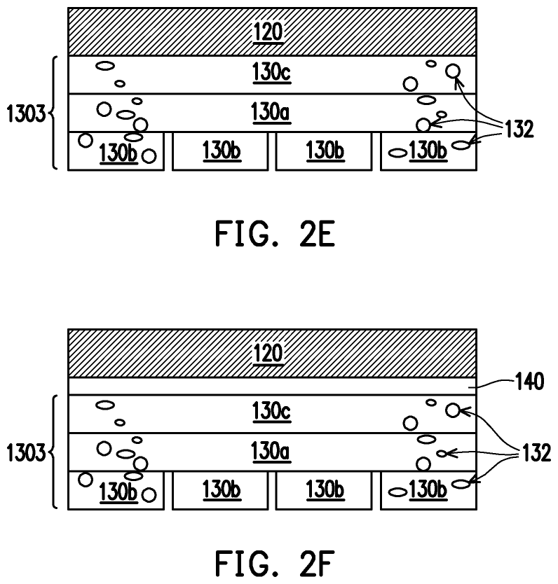

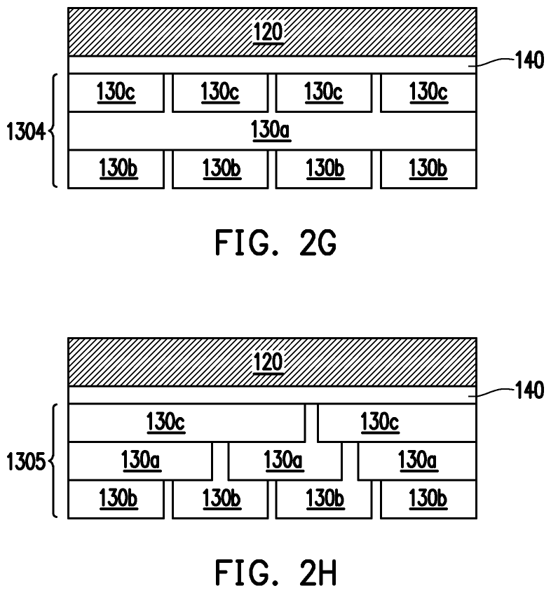

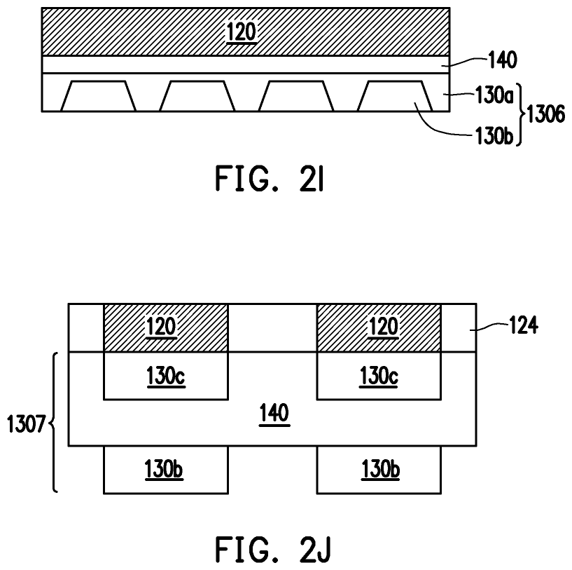

FIG. 2A to FIG. 2J are cross-sectional schematic diagrams of coupling sensing electrodes and coupling dielectric stacked layers according to various embodiments of the disclosure.

Referring to FIG. 2A, in this embodiment, the coupling dielectric stacked layer 130 includes a first dielectric layer 130a and a second dielectric layer 130b. The dielectric constant of the second dielectric layer 130b is greater than that of the first dielectric layer 130a, the first dielectric layer 130a covers the surface of the coupling sensing electrode 120, and the second dielectric layer 130b is disposed on the first dielectric layer 130a and is located between the first dielectric layer 130a and an organism (e.g., human skin S). In other words, the first dielectric layer 130a is located between the coupling sensing electrode 120 and the second dielectric layer 130b, and the first dielectric layer 130a is in contact with the coupling sensing electrode 120 and the second dielectric layer 130b. In some embodiments, the dielectric constant of the first dielectric layer 130a is between 3 and 7, the first dielectric layer 130a may be a silicon oxide (SiOₓ) layer, a silicon nitride (SiNₓ) layer, a silicon oxynitride layer (SiON) layer, or other suitable inorganic dielectric material layers, and the hardness of the first dielectric layer 130a is between 1 H and 4 H; the dielectric constant of the second dielectric layer 130b is between 7 and 10000, the second dielectric layer 130b may be a titanium oxide (TiOₓ) layer, an aluminum oxide (Al₂Oₓ) layer, a molybdenum nitride (MoN) layer, an aluminum nitride (AlN) layer, or other suitable inorganic dielectric material layer, and the hardness of the second dielectric layer 130b is between 5 H and 9 H. As mentioned above, the first dielectric layer 130a may play a role of a stress matching layer in the coupling dielectric stacked layer 130, and the second dielectric layer 130b may play a role of a coupling sensing layer in the coupling dielectric stacked layer 130. In some embodiments, the first dielectric layer 130a and the second dielectric layer 130b are both organic dielectric layers, and the organic dielectric layers include polyimide (PI) or other similar polymer materials. In the embodiment shown in FIG. 2A, the second dielectric layer 130b has properties of high dielectric constant, scratch resistance, abrasion resistance, and moisture resistance.

By adjusting the thickness and dielectric constant of the coupling dielectric stacked layer 130, the coupling capacitance between the coupling sensing electrode 120 and the organism (e.g., human skin S) may be increased.

In more detail, the capacitance C may be calculated and adjusted by the following formula:

$$C = \frac{{ε}_{0} A}{{h}_{1} \frac{1}{{ε}_{k 1}} + {h}_{2} \frac{1}{{ε}_{k 2}}}$$

In the above formula, each symbol is defined as follows:
A: The area of the coupling sensing electrode 120
hᵢ: thickness of the first dielectric layer 130a
h₂: the thickness of the second dielectric layer 130b
∈₀: 8.85×10⁻¹²
∈ₖ₁: the dielectric constant of the first dielectric layer 130a
∈ₖ₂: the dielectric constant of the second dielectric layer 130b

As shown in FIG. 2A, the first dielectric layer 130a and/or the second dielectric layer 130b may have pores 132. Since the pores 132 are randomly distributed, the pores 132 in the first dielectric layer 130a and the second dielectric layer 130b are respectively staggered, preventing moisture and/or corrosive gas from passing through the coupling dielectric stacked layer 130 to directly damage the coupling sensing electrodes 120, and the service life of the coupling sensing electrode 120 may be extended. Thereby, the reliability of the coupling sensing electrode 120 is improved.

As shown in FIG. 1A, the physiological sensing device 100 may further include a fabric layer 150, in which the second dielectric layer 130b is located between the first dielectric layer 130a and the fabric layer 150, and the fabric layer 150 is located between the organism (e.g., human skin S) and the second dielectric layer 130b.

Referring to FIG. 1B, the elements and details of the physiological sensing device 200 shown in FIG. 1B are substantially similar to the physiological sensing device 100 shown in FIG. 1A, therefore, the same reference numerals are used in FIG. 1B to denote the same or similar elements, and the description of the same technical content is omitted. Referring to FIG. 1A and FIG. 1B at the same time, the main difference between the physiological sensing device 200 shown in FIG. 1B and the physiological sensing device 100 shown in FIG. 1A is that the projected area of the noise isolation layer 122 covers the projected area of the coupling sensing electrode 120′. In other words, the projected area of the noise isolation layer 122 may be greater than or equal to the projected area of the coupling sensing electrodes 120′.

Referring to FIG. 2A and FIG. 2B, the cross-sectional schematic diagram of the coupling sensing electrode and the coupling dielectric stacked layer shown in FIG. 2B is similar to the cross-sectional schematic diagram of the coupling sensing electrode and the coupling dielectric stacked layer shown in FIG. 2A, but the main difference between the two is that the coupling dielectric stacked layer 1301 adopts a patterned design to reduce the problem of excessive stress in the coupling dielectric stacked layer 1301, thereby preventing peeling problems in the manufacturing process. The patterned second dielectric layer 130b facilitates in alleviating the problem that the coupling dielectric stacked layer 1301 cannot be bent, so that the coupling dielectric stacked layer 1301 has a bendable property. The properties such as the material of the coupling dielectric stacked layer 1301 are similar to the details (i.e., material, dielectric constant, thickness, etc.) of the coupling dielectric stacked layer 130 in FIG. 2A, and thus are not repeated herein.

In more detail, the capacitance C may be calculated and adjusted by the following formula:

$$C = P \% \times \frac{{ε}_{0} A}{{h}_{1} \frac{1}{{ε}_{k 1}} + {h}_{2} \frac{1}{{ε}_{k 2}}}$$

In the above formula, each symbol is defined as follows:
A: The area of the coupling sensing electrode 120
h₁: thickness of the first dielectric layer 130a
h₂: the thickness of the second dielectric layer 130b
ϵ₀: 8.85×10⁻¹²
ϵₖ₁: the dielectric constant of the first dielectric layer 130a
ϵₖ₂: the dielectric constant of the second dielectric layer 130b
P %: the area ratio of the second dielectric layer 130b to the coupling sensing electrode 120

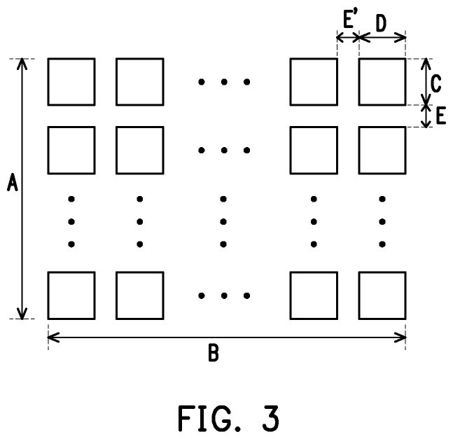

FIG. 3 is a schematic diagram of a pattern of a coupling dielectric stacked layer according to an embodiment of the disclosure. Referring to FIG. 3, the second dielectric layer 130b includes multiple rectangular dielectric patterns arranged in an array. The rectangular dielectric patterns are arranged in m columns and n rows, the rectangular dielectric patterns are arranged in a rectangular area, the dimension of the rectangular area in the column direction is A, and the dimension of the rectangular area in the row direction is B. The dimension of each rectangular dielectric pattern in the column direction is C, and the dimension of each rectangular dielectric pattern in the row direction is D. In addition, the spacing of the rectangular dielectric patterns in the column direction is E, and the spacing of the rectangular dielectric patterns in the row direction is E′.

In more detail, the aforementioned area ratio P % may be calculated and adjusted by the following formulas:

$$A \times B = \frac{P \% \times m \times n ( C \times D )}{100} ,$$

$$A = n \times C + ( n - 1 ) \times E ,$$

$$B = m \times D + ( m - 1 ) \times {E}^{′} .$$

Referring to FIG. 2C, in this embodiment, the coupling dielectric stacked layer 1302 includes a first dielectric layer 130a, a second dielectric layer 130b, and a third dielectric layer 130c. The dielectric constant of the second dielectric layer 130b and the third dielectric layer 130c are greater than that of the first dielectric layer 130a, the third dielectric layer 130c covers the surface of the coupling sensing electrode 120, the first dielectric layer 130a is disposed on the third dielectric layer 130c, and the second dielectric layer 130b is disposed on the first dielectric layer 130a. The first dielectric layer 130a is located between the third dielectric layer 130c and the second dielectric layer 130b, the second dielectric layer 130b is located between the first dielectric layer 130a and the organism (e.g., human skin S), and the third dielectric layer 130c is located between the coupling sensing electrode 120 and the first dielectric layer 130a. In other words, the first dielectric layer 130a is in contact with the third dielectric layer 130c and the second dielectric layer 130b, and the third dielectric layer 130c is in contact with the first dielectric layer 130a and the coupling sensing electrode 120. In some embodiments, the dielectric constant of the first dielectric layer 130a is between 3 and 7, the first dielectric layer 130a may be a silicon oxide (SiOₓ) layer, a silicon nitride (SiNₓ) layer, a silicon oxynitride layer (SiON) layer, or other suitable inorganic dielectric material layers, and the hardness of the first dielectric layer 130a is between 1 H and 4 H; the dielectric constant of the second dielectric layer 130b is between 7 and 10000, the second dielectric layer 130b may be a titanium oxide (TiOₓ) layer, an aluminum oxide (Al₂Oₓ) layer, a molybdenum nitride (MoN) layer, an aluminum nitride (AlN) layer, or other suitable inorganic dielectric material layer, and the hardness of the second dielectric layer 130b is between 5 H and 9 H; the dielectric constant of the third dielectric layer 130c is between 7 and 10000, the third dielectric layer 130c may be a titanium oxide (TiOₓ) layer, an aluminum oxide (Al₂Oₓ) layer, a molybdenum nitride (MoN) layer, an aluminum nitride (AlN) layer or other suitable inorganic dielectric material layer, and the hardness of the third dielectric layer 130c is between 5 H and 9 H. As mentioned above, the first dielectric layer 130a may play the role of a stress matching layer in the coupling dielectric stacked layer 1302, and the second dielectric layer 130b and the third dielectric layer 130c may play the role of a coupling sensing layer in the coupling dielectric stacked layer 1302. In the embodiment shown in FIG. 2C, the second dielectric layer 130b has properties of high dielectric constant, scratch resistance, abrasion resistance, and moisture resistance.

By adjusting the thickness and dielectric constant of the coupling dielectric stacked layer 1302, the coupling capacitance between the coupling sensing electrode 120 and the organism (e.g., human skin S) may be increased.

In more detail, the capacitance C may be calculated and adjusted by the following formula:

$$C = \frac{{ε}_{0} A}{{h}_{1} \frac{1}{{ε}_{k 1}} + {h}_{2} \frac{1}{{ε}_{k 2}} + {h}_{3} \frac{1}{{ε}_{k 3}}}$$

In the above formula, each symbol is defined as follows:
A: The area of the coupling sensing electrode 120
h1: thickness of the first dielectric layer 130a
h₂: the thickness of the second dielectric layer 130b
h₃: the thickness of the third dielectric layer 130c
ϵ₀: 8.85×10⁻¹²
ϵₖ₁: the dielectric constant of the first dielectric layer 130a
ϵₖ₂: the dielectric constant of the second dielectric layer 130b
ϵₖ₃: the dielectric constant of the third dielectric layer 130c

As shown in FIG. 2C, the first dielectric layer 130a, the second dielectric layer 130b, and/or the third dielectric layer 130c may have pores 132. Since the pores 132 are randomly distributed, the pores 132 in the first dielectric layer 130a, the second dielectric layer 130b, and the third dielectric layer 130c are respectively staggered, preventing moisture and/or corrosive gas from passing through the coupling dielectric stacked layer 1302 to directly damage the coupling sensing electrodes 120, and the service life of the coupling sensing electrode 120 may be extended. Thereby, the reliability of the coupling sensing electrode 120 is improved.

Referring to FIG. 2C and FIG. 2D, the cross-sectional schematic diagram of the coupling sensing electrode and the coupling dielectric stacked layer shown in FIG. 2D is similar to the cross-sectional schematic diagram of the coupling sensing electrode and the coupling dielectric stacked layer shown in FIG. 2C, but the main difference between the two is that a stress compensation layer 140 is disposed between the coupling sensing electrode 120 and the coupling dielectric stacked layer 1302 to reduce the stress accumulation problem between the coupling dielectric stacked layer 1302 and the coupling sensing electrode 120, thereby preventing peeling problems in the manufacturing process.

Referring to FIG. 2C and FIG. 2E, the cross-sectional schematic diagram of the coupling sensing electrode and the coupling dielectric stacked layer shown in FIG. 2E is similar to the cross-sectional schematic diagram of the coupling sensing electrode and the coupling dielectric stacked layer shown in FIG. 2C, but the main difference between the two is that the coupling dielectric stacked layer 1303 adopts a patterned design to reduce the problem of excessive stress in the coupling dielectric stacked layer 1303, thereby preventing peeling problems in the manufacturing process. The patterned second dielectric layer 130b facilitates in alleviating the problem that the coupling dielectric stacked layer 1303 cannot be bent, so that the coupling dielectric stacked layer 1303 has a bendable property. Materials and other properties of the coupling dielectric stacked layer 1303 are not repeated herein.

Referring to FIG. 2D and FIG. 2F, the cross-sectional schematic diagram of the coupling sensing electrode and the coupling dielectric stacked layer shown in FIG. 2F is similar to the cross-sectional schematic diagram of the coupling sensing electrode and the coupling dielectric stacked layer shown in FIG. 2D, but the main difference between the two is that the coupling dielectric stacked layer 1303 adopts a patterned design to reduce the problem of excessive stress in the coupling dielectric stacked layer 1303, thereby preventing peeling problems in the manufacturing process. The patterned second dielectric layer 130b facilitates in alleviating the problem that the coupling dielectric stacked layer 1303 cannot be bent, so that the coupling dielectric stacked layer 1303 has a bendable property. Materials and other properties of the coupling dielectric stacked layer 1303 are not repeated herein.

Referring to FIG. 2F and FIG. 2G, the cross-sectional schematic diagram of the coupling sensing electrode and the coupling dielectric stacked layer shown in FIG. 2G is similar to the cross-sectional schematic diagram of the coupling sensing electrode and the coupling dielectric stacked layer shown in FIG. 2F, but the main difference between the two is that the second dielectric layer 130b and the third dielectric layer 130c in the coupling dielectric stacked layer 1304 all adopt a patterned design to reduce the problem of excessive stress in the coupling dielectric stacked layer 1304, thereby preventing peeling problems in the manufacturing process. The first dielectric layer 130a is filled between multiple separated dielectric patterns of the third dielectric layer 130c, and the third dielectric layer 130c may be separated from the second dielectric layer 130b by the first dielectric layer 130a. The patterned second dielectric layer 130b and third dielectric layer 130c facilitate in alleviating the problem that the coupling dielectric stacked layer 1304 cannot be bent, so that the coupling dielectric stacked layer 1304 has a bendable property. Materials and other properties of the coupling dielectric stacked layer 1304 are not repeated herein.

Referring to FIG. 2F and FIG. 2H, the cross-sectional schematic diagram of the coupling sensing electrode and the coupling dielectric stacked layer shown in FIG. 2H is similar to the cross-sectional schematic diagram of the coupling sensing electrode and the coupling dielectric stacked layer shown in FIG. 2F, but the main difference between the two is that the first dielectric layer 130a, the second dielectric layer 130b, and the third dielectric layer 130c in the coupling dielectric stacked layer 1305 all adopt a patterned design to reduce the problem of excessive stress in the coupling dielectric stacked layer 1305, thereby preventing peeling problems in the manufacturing process. The first dielectric layer 130a is filled between multiple separated dielectric patterns of the third dielectric layer 130c. The second dielectric layer 130b is filled between multiple separated dielectric patterns of the first dielectric layer 130a. In addition, the second dielectric layer 130b may be in contact with the third dielectric layer 130c. The patterned first dielectric layer 130a, second dielectric layer 130b, and third dielectric layer 130c facilitate in alleviating the problem that the coupling dielectric stacked layer 1305 cannot be bent, so that the coupling dielectric stacked layer 1305 has a bendable property. Materials and other properties of the coupling dielectric stacked layer 1305 are not repeated herein.

Referring to FIG. 2B and FIG. 2I, the cross-sectional schematic diagram of the coupling sensing electrode and the coupling dielectric stacked layer shown in FIG. 2I is similar to the cross-sectional schematic diagram of the coupling sensing electrode and the coupling dielectric stacked layer shown in FIG. 2B, but the main difference between the two is that the second dielectric layer 130b in the coupling dielectric stacked layer 1306 is embedded in the first dielectric layer 130a, and the outer surfaces of the first dielectric layer 130a and the second dielectric layer 130b are substantially coplanar. Since the second dielectric layer 130b adopts a patterned design, the problem of excessive stress in the coupling dielectric stacked layer 1306 is reduced, thereby preventing peeling problems in the manufacturing process. The patterned second dielectric layer 130b facilitates in alleviating the problem that the coupling dielectric stacked layer 1306 cannot be bent, so that the coupling dielectric stacked layer 1306 has a bendable property. Materials and other properties of the coupling dielectric stacked layer 1306 are not repeated herein.

Referring to FIG. 2G and FIG. 2J, the cross-sectional schematic diagram of the coupling sensing electrode and the coupling dielectric stacked layer shown in FIG. 2J is similar to the cross-sectional schematic diagram of the coupling sensing electrode and the coupling dielectric stacked layer shown in FIG. 2G, but the main difference between the two is that the stress compensation layer 140 shown in FIG. 2J is used to replace the first dielectric layer 130a shown in FIG. 2G, and a patterned coupling sensing electrode 120 is adopted, in which the patterned coupling sensing electrode 120 is embedded in the dielectric layer 124. In some embodiments, the coupling sensing electrode 120 includes a meshed electrode pattern embedded in the dielectric layer 124. Since the coupling sensing electrode 120, the second dielectric layer 130b, and the third dielectric layer 130c all adopt a patterned design, the problem of excessive stress of the coupling dielectric stacked layer 1307 may be reduced, thereby preventing peeling problems in the manufacturing process. The patterned coupling sensing electrode 120, second dielectric layer 130b, and third dielectric layer 130c facilitate in alleviating the problem that the coupling dielectric stacked layer 1307 cannot be bent, so that the coupling dielectric stacked layer 1307 has a bendable property. Materials and other properties of the coupling dielectric stacked layer 1307 are not repeated herein.

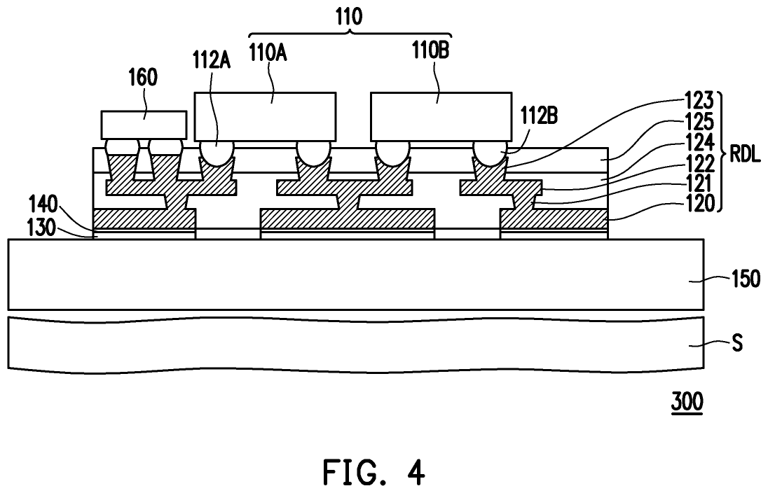

FIG. 4 is a cross-sectional schematic diagram of a physiological sensing device according to a second embodiment of the disclosure. Referring to FIG. 1A and FIG. 4, the physiological sensing device 300 shown in FIG. 4 is similar to the physiological sensing device 100 shown in FIG. 1A, but the main difference between the two is that the physiological sensing device 300 of this embodiment further includes a passive component 160, in which the passive component 160 is disposed on the coupling stress adjustment layer 125 and is electrically connected to the redistribution layer RDL. In this embodiment, the passive component 160 is electrically connected to the redistribution layer RDL through solder bonding.

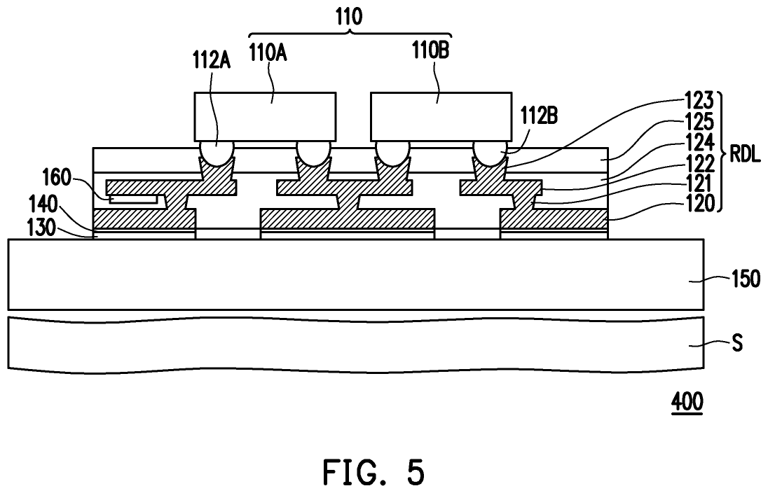

FIG. 5 is a cross-sectional schematic diagram of a physiological sensing device according to a third embodiment of the disclosure. Referring to FIG. 5, the physiological sensing device 400 shown in FIG. 5 is similar to the physiological sensing device 100 shown in FIG. 1A, but the main difference between the two is that the physiological sensing device 400 of this embodiment further includes a passive component 160, in which the passive component 160 is disposed in the redistribution layer RDL, and is electrically connected to the redistribution layer RDL. In this embodiment, the fabrication of the passive component 160 may be integrated with the fabrication of the redistribution layer RDL.

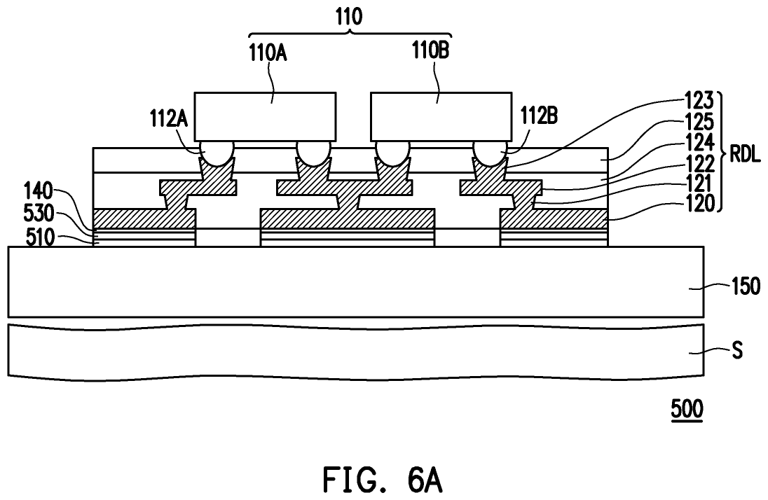

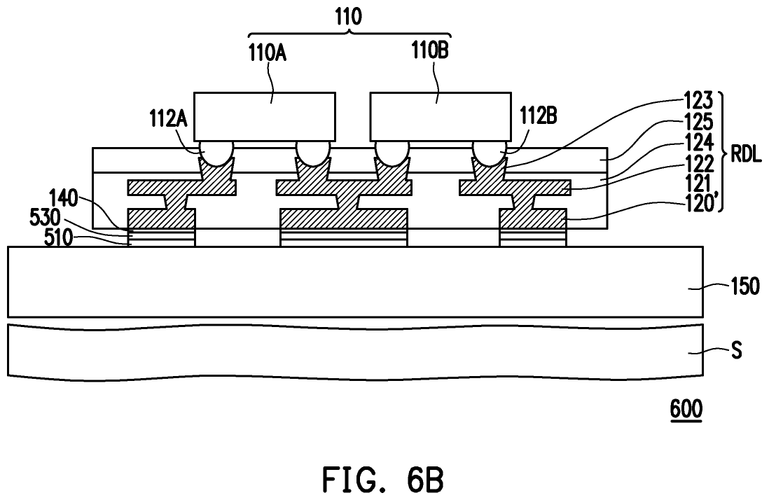

FIG. 6A and FIG. 6B are cross-sectional schematic diagrams of a physiological sensing device according to a fourth embodiment of the disclosure.

Referring to FIG. 6A, the physiological sensing device 500 of the embodiment is suitable for sensing physiological signal of an organism (e.g., humans or pets), such as electromyography signals, electrocardiography signals, or electroencephalography signals. The physiological sensing device 500 of this embodiment includes at least one sensing chip 110, a coupling sensing electrode 120, a conductive layer 510, and a coupling dielectric stacked layer 530. The coupling sensing electrode 120 is electrically connected to the sensing chip 110, the coupling dielectric stacked layer 530 covers the coupling sensing electrode 120, the conductive layer 510 covers the coupling dielectric stacked layer 530, and the conductive layer 510 and the coupling dielectric stacked layer 530 are located between the coupling sensing electrode 120 and the organism (e.g., human skin S). As shown in FIG. 6A, the coupling dielectric stacked layer 530 is located between the coupling sensing electrode 120 and the conductive layer 510, and the conductive layer 510 is electrically insulated from the coupling sensing electrode 120 by the coupling dielectric stacked layer 530. In some embodiments, the conductive layer 510 is electrically floating, and the conductive layer 510 and the coupling sensing electrode are respectively located on two opposite sides of the coupling dielectric stacked layer 530. In this embodiment, the sensing chip 110 include a first sensing chip 110A and a second sensing chip 110B. However, the number of the sensing chips 110 is for illustration only, and is not intended to limit the number of the sensing chips 110 used in the disclosure. In some embodiments, the first sensing chip 110A includes a high impedance front end circuit module, and the second sensing chip 110B includes a sensing integrated circuit. As shown in FIG. 6A, the first sensing chip 110A may have a multiple conductive terminals 112A, and the second sensing chip 110B may have multiple conductive terminals 112B, and the conductive terminals 112A and 112B are, for example, solder balls.

In some embodiments, the coupling sensing electrodes 120 may be integrated into a redistribution layer RDL with flexibility, and the redistribution layer RDL may include the coupling sensing electrode 120, a conductive via 121, a noise isolation layer 122, a wire layer 123, multiple dielectric layers 124, and a coupling stress adjustment layer 125. The coupling sensing electrode 120, the conductive via 121, and the noise isolation layer 122 are embedded in the dielectric layers 124, and the coupling sensing electrode 120 is electrically connected to the noise isolation layer 122 through the conductive via 121. In addition, the physiological sensing device 500 may further include a stress compensation layer 140, in which the stress compensation layer 140 is disposed between the coupling sensing electrode 120 and the coupling dielectric stacked layer 530. The stress compensation layer 140 may be formed by a chemical vapor deposition process. By controlling the parameters of the chemical vapor deposition process, the stress compensation layer 140 with a suitable stress value (tensile stress or compressive stress) may be fabricated. The material of the stress compensation layer 140 may be an inorganic material and may include, for example, Pb(ZrTi)O₃, SiO₂, ZnO, Ta₂O₅, Si₃N₄, SiON, BaTiO₃, CaTiO₃, SrTiO₃, TiO₂, MgO, AN, or Al₂O₃.

In this embodiment, the stress value of the coupling dielectric stacked layer 530 may be adjusted in consideration of the overall stress value of the physiological sensing device 500. For example, when the stress value of the physiological sensing device 500 is, for example, 50 Mpa to 200 Mpa (tensile stress), which causes the physiological sensing device 500 to curl, the stress value of the coupling dielectric stacked layer 530 may be adjusted to, for example, −50 Mpa to −200 Mpa to flatten the physiological sensing device 500; when the stress value of the physiological sensing device 500 is, for example, −50 Mpa to −200 Mpa (compressive stress), which causes the physiological sensing device 500 to curl, the stress value of the coupling dielectric stacked layer 530 may be adjusted to, for example, 50 Mpa to 200 Mpa to flatten the physiological sensing device 500.

In this embodiment, the stress compensation layer 140 may improve the stress matching between the coupling dielectric stacked layer 530 and other film layers (e.g., the coupling sensing electrode 120, the noise isolation layer 122, the wire layer 123, etc.) to prevent peeling problems in the manufacturing process. In this embodiment, the stress value of the stress compensation layer 140 may be adjusted in consideration of the stress value of the physiological sensing device 500. For example, when the stress value of the physiological sensing device 500 is, for example, 50 Mpa to 200 Mpa (tensile stress), which causes the physiological sensing device 500 to curl, the stress value of the stress compensation layer 140 may be adjusted to, for example, −50 Mpa to −−200 Mpa to flatten the physiological sensing device 500; when the stress value of the physiological sensing device 500 is, for example, −50 Mpa to −200 Mpa (compressive stress), which causes the physiological sensing device 500 to curl, the stress value of the stress compensation layer 140 may be adjusted to, for example, 50 Mpa to 200 Mpa to flatten the physiological sensing device 500. At the same time, the coupling capacitance may still maintain at 1 nF to 10 nF.

It should be noted that the coupling dielectric stacked layer 530 and the stress compensation layer 140 may individually be used to adjust the overall stress of the physiological sensing device 500 or simultaneously be used to adjust the overall stress of the physiological sensing device 500.

The first sensing chip 110A and the second sensing chip 110B may be respectively electrically connected to the redistribution layer RDL below the conductive terminal 112A and the conductive terminal 112B through the conductive terminal 112A and the conductive terminal 112B. Specifically, the first sensing chip 110A may be electrically connected to the coupling sensing electrodes 120 through the conductive terminal 112A, the conductive terminal 112B, the wire layer 123, and the conductive via 121.

The coupling sensing electrode 120 in the redistribution layer RDL is used to sense the physiological signal of the organism. During the sensing of the physiological signal, capacitance is generated between the organism and the coupling sensing electrodes 120, in which the organism is, for example, human skin S. The coupling dielectric stacked layer 530 is disposed below the coupling sensing electrode 120. The noise isolation layer 122 is disposed above the coupling sensing electrode 120, and the coupling sensing electrode 120 and the noise isolation layer 122 overlap each other in the vertical projection direction. In this embodiment, the noise isolation layer 122 may include a ground circuit or a ground patch. The noise isolation layer 122 may prevent the coupling sensing electrodes 120 from directly receiving external noise, resulting in the signal-to-noise ratio (SNR) of the physiological signal being too small to be accurately sensed. The wire layer 123 is disposed above the noise isolation layer 122, and the wire layer 123 is electrically connected to the first sensing chip 110A and the second sensing chip 110B, and the coupling sensing electrodes 120. The wire layer 123 is respectively electrically connected to the first sensing chip 110A and the second sensing chip 110B, for example, through the conductive terminal 112A and the conductive terminal 112B. The layout design of the redistribution layer RDL may be adjusted according to design requirements, which is not limited in the disclosure. The coupling stress adjustment layer 125 is disposed above the dielectric layer 124, and the coupling stress adjustment layer 125 may be used to improve the overall stress matching of the physiological sensing device 500. In some embodiments of the disclosure, the physiological sensing device 500 may further include an insulating encapsulation layer (not shown). The insulating encapsulation layer is disposed above the coupling stress adjusting layer 125 and covers the first sensing chip 110A and the second sensing chip 110B. The insulating encapsulation layer may be filled in the gap between the coupling stress adjustment layer 125 and the first sensing chip 110A and the gap between the coupling stress adjustment layer 125 and the second sensing chip 110B to further laterally cover the conductive terminal 112A and the conductive terminal 112B. In addition, the material of the insulating encapsulation layer is, for example, a soft material, which may include polydimethylsiloxane (PDMS) or thermoplastic polyurethane (TPU), which is used to provide protection and insulation for the first sensing chip 110A and the second sensing chip 110B.

In this embodiment, the material of the coupling sensing electrode 120 may include Mo, Ti, Al, or Cu, and the thickness of the coupling sensing electrode 120 is, for example, 300 nm to 5000 nm. In this embodiment, the material of the conductive layer 510 and the coupling sensing electrode 120 may be the same or different. For example, the material of the conductive layer 510 may include Mo, Ti, Al, Cu, etc., or transparent conductive materials, such as indium tin oxide (ITO), indium zinc oxide (IZO), indium gallium zinc oxide (IGZO), etc.

As shown in FIG. 6A, the physiological sensing device 500 may further include a fabric layer 150, in which the conductive layer 510 is located between the coupling dielectric stacked layer 530 and the fabric layer 150, and the fabric layer 150 is located between the organism (e.g., human skin S) and the coupling dielectric stacked layer 530. In the embodiment where the physiological sensing device 500 further includes the fabric layer 150, the capacitor formed by the conductors on two sides of the fabric layer 150 (i.e., the skin of the organism and the conductive layer 510) is connected in parallel with the capacitor formed by the conductors on two sides of the coupling dielectric stacked layer 530 (i.e., the conductive layer 510 and the coupling sensing electrode 120), and therefore, the aforementioned two parallel capacitors may form a higher equivalent capacitance. Accordingly, the conductive layer 510 facilitates in further improving the sensing sensitivity of the physiological sensing device 500.

In the embodiment where the physiological sensing device 500 further includes the fabric layer 150, compared with the physiological sensing device without the conductive layer 510, the physiological sensing device 500 with the conductive layer 510 may improve the signal-to-noise ratio (SNR) by about 129%, the physiological sensing device 500 with the conductive layer 510 may improve the feedback voltage by about 22%, and the physiological sensing device 500 with the conductive layer 510 may improve noise suppression by about 56%.

In the embodiment where the physiological sensing device 500 does not further include the fabric layer 150, compared with the physiological sensing device without the conductive layer 510, the physiological sensing device 500 with the conductive layer 510 may improve the signal-to-noise ratio (SNR) by about 25%, the physiological sensing device 500 with the conductive layer 510 may improve the feedback voltage by about 31%, and the physiological sensing device 500 with the conductive layer 510 may improve noise suppression by about 12%.

Referring to FIG. 6B, the elements and details of the physiological sensing device 600 shown in FIG. 6B are substantially similar to the physiological sensing device 500 shown in FIG. 6A, therefore, the same reference numerals are used in FIG. 6B to denote the same or similar elements, and the description of the same technical content is omitted. Referring to FIG. 6A and FIG. 6B at the same time, the main difference between the physiological sensing device 600 shown in FIG. 6B and the physiological sensing device 500 shown in FIG. 6A is that the projected area of the noise isolation layer 122 covers the projected area of the coupling sensing electrode 120′. In other words, the projected area of the noise isolation layer 122 may be greater than or equal to the projected area of the coupling sensing electrodes 120′.

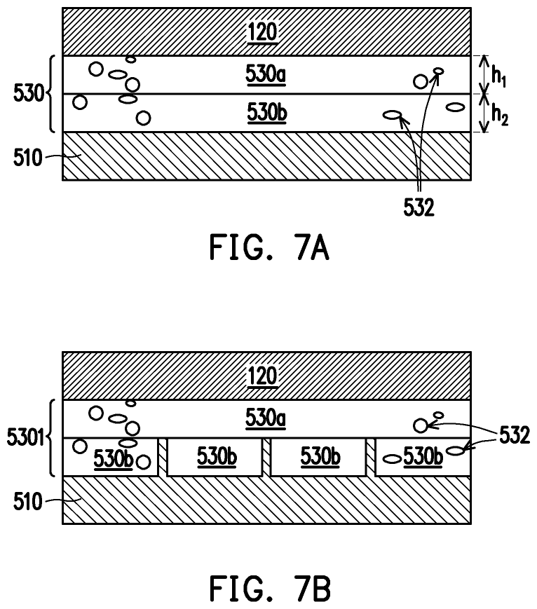

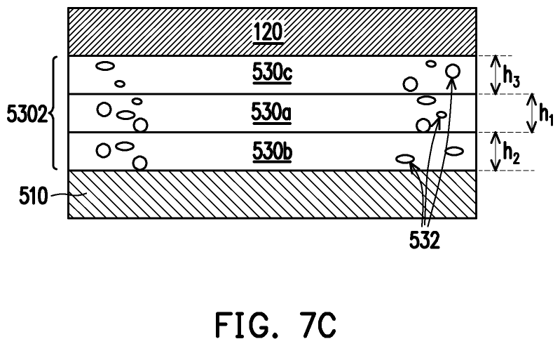

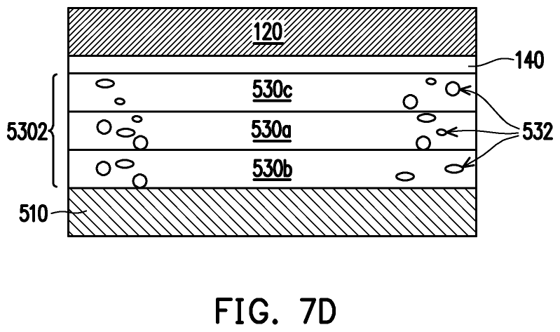

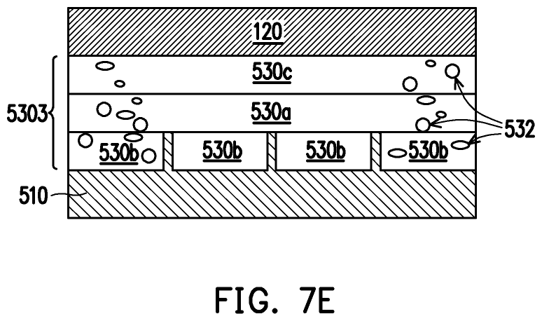

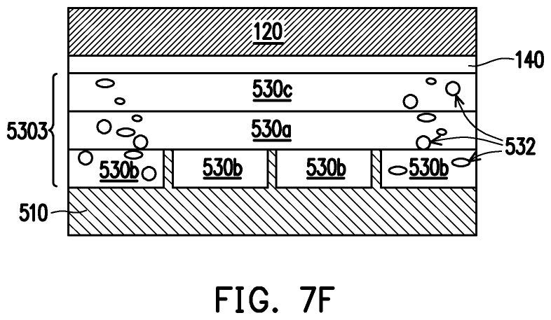

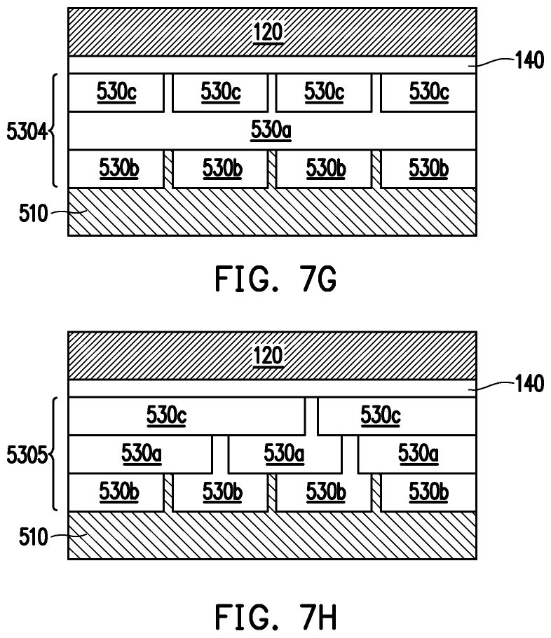

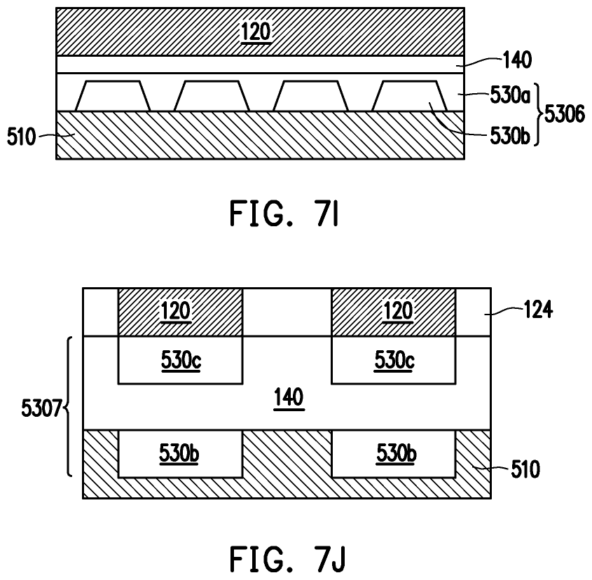

FIG. 7A to FIG. 7J are cross-sectional schematic diagrams of coupling sensing electrodes, coupling dielectric stacked layers, and conductive layers according to various embodiments of the disclosure.

Referring to FIG. 7A, in this embodiment, the coupling dielectric stacked layer 530 includes a first dielectric layer 530a and a second dielectric layer 530b. The dielectric constant of the second dielectric layer 530b is greater than that of the first dielectric layer 530a, the first dielectric layer 530a covers the surface of the coupling sensing electrode 120, and the second dielectric layer 530b is disposed on the first dielectric layer 530a and is located between the first dielectric layer 530a and an organism (e.g., human skin S). In other words, the first dielectric layer 530a is located between the coupling sensing electrode 120 and the second dielectric layer 530b, and the first dielectric layer 530a is in contact with the coupling sensing electrode 120 and the second dielectric layer 530b. In some embodiments, the dielectric constant of the first dielectric layer 530a is between 3 and 7, the first dielectric layer 530a may be a silicon oxide (SiOₓ) layer, a silicon nitride (SiNₓ) layer, a silicon oxynitride layer (SiON) layer, or other suitable inorganic dielectric material layers, and the hardness of the first dielectric layer 530a is between 1 H and 4 H; the dielectric constant of the second dielectric layer 530b is between 7 and 10000, the second dielectric layer 530b may be a titanium oxide (TiOₓ) layer, an aluminum oxide (Al₂Oₓ) layer, a molybdenum nitride (MoN) layer, an aluminum nitride (AlN) layer, or other suitable inorganic dielectric material layer, and the hardness of the second dielectric layer 530b is between 5 H and 9 H. As mentioned above, the first dielectric layer 530a may play a role of a stress matching layer in the coupling dielectric stacked layer 530, and the second dielectric layer 530b may play a role of a coupling sensing layer in the coupling dielectric stacked layer 530. In some embodiments, the first dielectric layer 530a and the second dielectric layer 530b are both organic dielectric layers, and the organic dielectric layers include polyimide (PI) or other similar polymer materials. In the embodiment shown in FIG. 7A, the second dielectric layer 530b has properties of high dielectric constant, scratch resistance, abrasion resistance, and moisture resistance.

By adjusting the thickness and dielectric constant of the coupling dielectric stacked layer 530, the coupling capacitance between the coupling sensing electrode 120 and the organism (e.g., human skin S) may be increased. In addition, the conductive layer 510 covers the second dielectric layer 530b, and the second dielectric layer 530b is located between the first dielectric layer 530a and the conductive layer 510 to separate the first dielectric layer 530a from the conductive layer 510.

As shown in FIG. 7A, the first dielectric layer 530a and/or the second dielectric layer 130b may have pores 532. Since the pores 532 are randomly distributed, the pores 532 in the first dielectric layer 530a and the second dielectric layer 530b are respectively staggered, preventing moisture and/or corrosive gas from passing through the coupling dielectric stacked layer 530 to directly damage the coupling sensing electrodes 120, and the service life of the coupling sensing electrode 120 may be extended. Thereby, the reliability of the coupling sensing electrode 120 is improved.

Referring to FIG. 7A and FIG. 7B, the cross-sectional schematic diagram of the coupling sensing electrode and the coupling dielectric stacked layer shown in FIG. 7B is similar to the cross-sectional schematic diagram of the coupling sensing electrode and the coupling dielectric stacked layer shown in FIG. 7A, but the main difference between the two is that the coupling dielectric stacked layer 5301 adopts a patterned design to reduce the problem of excessive stress in the coupling dielectric stacked layer 5301, thereby preventing peeling problems in the manufacturing process. The patterned second dielectric layer 530b facilitates in alleviating the problem that the coupling dielectric stacked layer 5301 cannot be bent, so that the coupling dielectric stacked layer 5301 has a bendable property. The properties such as the material of the coupling dielectric stacked layer 5301 are similar to the details (i.e., material, dielectric constant, thickness, etc.) of the coupling dielectric stacked layer 530 in FIG. 7A, and thus are not repeated herein.

Referring to FIG. 7C, in this embodiment, the coupling dielectric stacked layer 5302 includes a first dielectric layer 530a, a second dielectric layer 530b, and a third dielectric layer 530c. The dielectric constant of the second dielectric layer 530b and the third dielectric layer 530c are greater than that of the first dielectric layer 530a, the third dielectric layer 530c covers the surface of the coupling sensing electrode 120, the first dielectric layer 530a is disposed on the third dielectric layer 530c, and the second dielectric layer 530b is disposed on the first dielectric layer 530a. The first dielectric layer 530a is located between the third dielectric layer 530c and the second dielectric layer 530b, the second dielectric layer 530b is located between the first dielectric layer 530a and the organism (e.g., human skin S), and the third dielectric layer 530c is located between the coupling sensing electrode 120 and the first dielectric layer 530a. In other words, the first dielectric layer 530a is in contact with the third dielectric layer 530c and the second dielectric layer 530b, and the third dielectric layer 530c is in contact with the first dielectric layer 530a and the coupling sensing electrode 120. In some embodiments, the dielectric constant of the first dielectric layer 530a is between 3 and 7, the first dielectric layer 530a may be a silicon oxide (SiOₓ) layer, a silicon nitride (SiNₓ) layer, a silicon oxynitride layer (SiON) layer, or other suitable inorganic dielectric material layers, and the hardness of the first dielectric layer 530a is between 1 H and 4 H; the dielectric constant of the second dielectric layer 530b is between 7 and 10000, the second dielectric layer 530b may be a titanium oxide (TiOₓ) layer, an aluminum oxide (Al₂Oₓ) layer, a molybdenum nitride (MoN) layer, an aluminum nitride (AlN) layer, or other suitable inorganic dielectric material layer, and the hardness of the second dielectric layer 530b is between 5 H and 9 H; the dielectric constant of the third dielectric layer 530c is between 7 and 10000, the third dielectric layer 530c may be a titanium oxide (TiOₓ) layer, an aluminum oxide (Al₂Oₓ) layer, a molybdenum nitride (MoN) layer, an aluminum nitride (AlN) layer or other suitable inorganic dielectric material layer, and the hardness of the third dielectric layer 530c is between 5 H and 9 H. As mentioned above, the first dielectric layer 530a may play the role of a stress matching layer in the coupling dielectric stacked layer 5302, and the second dielectric layer 530b and the third dielectric layer 530c may play the role of a coupling sensing layer in the coupling dielectric stacked layer 5302. In the embodiment shown in FIG. 7C, the second dielectric layer 530b has properties of high dielectric constant, scratch resistance, abrasion resistance, and moisture resistance.

By adjusting the thickness and dielectric constant of the coupling dielectric stacked layer 5302, the coupling capacitance between the coupling sensing electrode 120 and the organism (e.g., human skin S) may be increased.

As shown in FIG. 7C, the first dielectric layer 530a, the second dielectric layer 530b, and/or the third dielectric layer 530c may have pores 532. Since the pores 532 are randomly distributed, the pores 532 in the first dielectric layer 530a, the second dielectric layer 530b, and the third dielectric layer 530c are respectively staggered, preventing moisture and/or corrosive gas from passing through the coupling dielectric stacked layer 5302 to directly damage the coupling sensing electrodes 120, and the service life of the coupling sensing electrode 120 may be extended. Thereby, the reliability of the coupling sensing electrode 120 is improved.

Referring to FIG. 7C and FIG. 7D, the cross-sectional schematic diagram of the coupling sensing electrode and the coupling dielectric stacked layer shown in FIG. 7D is similar to the cross-sectional schematic diagram of the coupling sensing electrode and the coupling dielectric stacked layer shown in FIG. 7C, but the main difference between the two is that a stress compensation layer 140 is disposed between the coupling sensing electrode 120 and the coupling dielectric stacked layer 5302 to reduce the stress accumulation problem between the coupling dielectric stacked layer 5302 and the coupling sensing electrode 120, thereby preventing peeling problems in the manufacturing process.

Referring to FIG. 7C and FIG. 7E, the cross-sectional schematic diagram of the coupling sensing electrode and the coupling dielectric stacked layer shown in FIG. 7E is similar to the cross-sectional schematic diagram of the coupling sensing electrode and the coupling dielectric stacked layer shown in FIG. 7C, but the main difference between the two is that the coupling dielectric stacked layer 5303 adopts a patterned design to reduce the problem of excessive stress in the coupling dielectric stacked layer 5303, thereby preventing peeling problems in the manufacturing process. The patterned second dielectric layer 530b facilitates in alleviating the problem that the coupling dielectric stacked layer 5303 cannot be bent, so that the coupling dielectric stacked layer 5303 has a bendable property. Materials and other properties of the coupling dielectric stacked layer 5303 are not repeated herein.

Referring to FIG. 7D and FIG. 7F, the cross-sectional schematic diagram of the coupling sensing electrode and the coupling dielectric stacked layer shown in FIG. 7F is similar to the cross-sectional schematic diagram of the coupling sensing electrode and the coupling dielectric stacked layer shown in FIG. 7D, but the main difference between the two is that the coupling dielectric stacked layer 5303 adopts a patterned design to reduce the problem of excessive stress in the coupling dielectric stacked layer 5303, thereby preventing peeling problems in the manufacturing process. The patterned second dielectric layer 530b facilitates in alleviating the problem that the coupling dielectric stacked layer 5303 cannot be bent, so that the coupling dielectric stacked layer 5303 has a bendable property. Materials and other properties of the coupling dielectric stacked layer 5303 are not repeated herein.

Referring to FIG. 7F and FIG. 7G, the cross-sectional schematic diagram of the coupling sensing electrode and the coupling dielectric stacked layer shown in FIG. 7G is similar to the cross-sectional schematic diagram of the coupling sensing electrode and the coupling dielectric stacked layer shown in FIG. 7F, but the main difference between the two is that the second dielectric layer 530b and the third dielectric layer 530c in the coupling dielectric stacked layer 5304 all adopt a patterned design to reduce the problem of excessive stress in the coupling dielectric stacked layer 5304, thereby preventing peeling problems in the manufacturing process. The first dielectric layer 530a is filled between multiple separated dielectric patterns of the third dielectric layer 530c, and the third dielectric layer 530c may be separated from the second dielectric layer 530b by the first dielectric layer 530a. The patterned second dielectric layer 530b and third dielectric layer 530c facilitate in alleviating the problem that the coupling dielectric stacked layer 5304 cannot be bent, so that the coupling dielectric stacked layer 5304 has a bendable property. Materials and other properties of the coupling dielectric stacked layer 5304 are not repeated herein.

Referring to FIG. 7F and FIG. 7H, the cross-sectional schematic diagram of the coupling sensing electrode and the coupling dielectric stacked layer shown in FIG. 7H is similar to the cross-sectional schematic diagram of the coupling sensing electrode and the coupling dielectric stacked layer shown in FIG. 7F, but the main difference between the two is that the first dielectric layer 530a, the second dielectric layer 530b, and the third dielectric layer 530c in the coupling dielectric stacked layer 5305 all adopt a patterned design to reduce the problem of excessive stress in the coupling dielectric stacked layer 5305, thereby preventing peeling problems in the manufacturing process. The first dielectric layer 530a is filled between multiple separated dielectric patterns of the third dielectric layer 530c. The second dielectric layer 530b is filled between multiple separated dielectric patterns of the first dielectric layer 530a. In addition, the second dielectric layer 530b may be in contact with the third dielectric layer 530c. The patterned first dielectric layer 530a, second dielectric layer 530b, and third dielectric layer 530c facilitate in alleviating the problem that the coupling dielectric stacked layer 5305 cannot be bent, so that the coupling dielectric stacked layer 5305 has a bendable property. Materials and other properties of the coupling dielectric stacked layer 5305 are not repeated herein.

Referring to FIG. 7B and FIG. 7I, the cross-sectional schematic diagram of the coupling sensing electrode and the coupling dielectric stacked layer shown in FIG. 7I is similar to the cross-sectional schematic diagram of the coupling sensing electrode and the coupling dielectric stacked layer shown in FIG. 7B, but the main difference between the two is that the second dielectric layer 530b in the coupling dielectric stacked layer 5306 is embedded in the first dielectric layer 530a, and the outer surfaces of the first dielectric layer 530a and the second dielectric layer 530b are substantially coplanar. Since the second dielectric layer 530b adopts a patterned design, the problem of excessive stress in the coupling dielectric stacked layer 5306 is reduced, thereby preventing peeling problems in the manufacturing process. The patterned second dielectric layer 530b facilitates in alleviating the problem that the coupling dielectric stacked layer 5306 cannot be bent, so that the coupling dielectric stacked layer 5306 has a bendable property. Materials and other properties of the coupling dielectric stacked layer 5306 are not repeated herein.

Referring to FIG. 7G and FIG. 7J, the cross-sectional schematic diagram of the coupling sensing electrode and the coupling dielectric stacked layer shown in FIG. 7J is similar to the cross-sectional schematic diagram of the coupling sensing electrode and the coupling dielectric stacked layer shown in FIG. 7G, but the main difference between the two is that the stress compensation layer 140 shown in FIG. 7J is used to replace the first dielectric layer 530a shown in FIG. 7G, and a patterned coupling sensing electrode 120 is adopted, in which the patterned coupling sensing electrode 120 is embedded in the dielectric layer 124. In some embodiments, the coupling sensing electrode 120 includes a meshed electrode pattern embedded in the dielectric layer 124. Since the coupling sensing electrode 120, the second dielectric layer 530b, and the third dielectric layer 530c all adopt a patterned design, the problem of excessive stress of the coupling dielectric stacked layer 5307 may be reduced, thereby preventing peeling problems in the manufacturing process. The patterned coupling sensing electrode 120, second dielectric layer 530b, and third dielectric layer 530c facilitate in alleviating the problem that the coupling dielectric stacked layer 5307 cannot be bent, so that the coupling dielectric stacked layer 5307 has a bendable property. Materials and other properties of the coupling dielectric stacked layer 5307 are not repeated herein.

It should be noted that the aforementioned coupling dielectric stacked layers 530, 5301, 5302, 5303, 5304, 5305, 5306, and 5307 may be replaced by a single-layer coupling dielectric layer (e.g., a single-material dielectric layer). In these embodiments, the coupling sensing electrode 120, the single-layer dielectric layer and the conductive layer 510 may also achieve the effect of further increasing the sensing sensitivity.

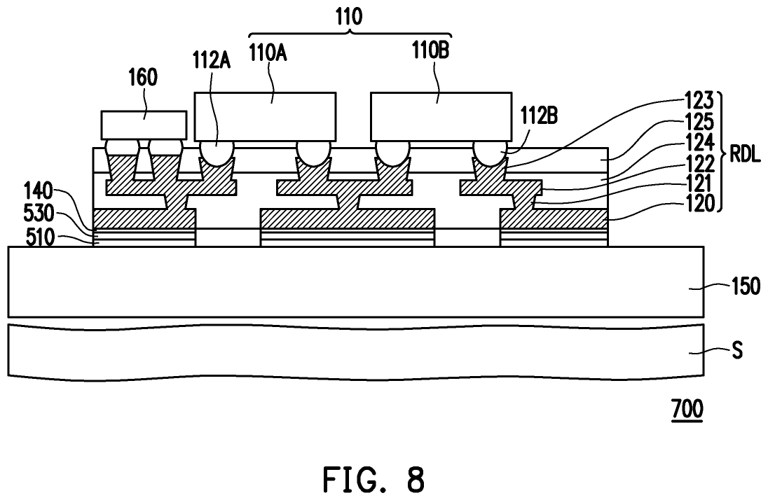

FIG. 8 is a cross-sectional schematic diagram of a physiological sensing device according to a fifth embodiment of the disclosure. Referring to FIG. 6A and FIG. 8, the physiological sensing device 700 shown in FIG. 8 is similar to the physiological sensing device 500 shown in FIG. 6A, but the main difference between the two is that the physiological sensing device 700 of this embodiment further includes a passive component 160, in which the passive component 160 is disposed on the coupling stress adjustment layer 125 and is electrically connected to the redistribution layer RDL. In this embodiment, the passive component 160 is electrically connected to the redistribution layer RDL through solder bonding.

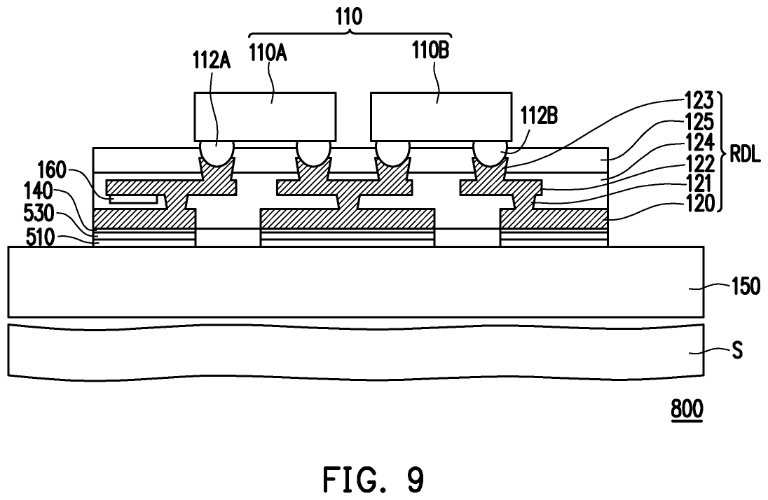

FIG. 9 is a cross-sectional schematic diagram of a physiological sensing device according to a sixth embodiment of the disclosure. Referring to FIG. 6A and FIG. 9, the physiological sensing device 800 shown in FIG. 9 is similar to the physiological sensing device 500 shown in FIG. 6A, but the main difference between the two is that the physiological sensing device 800 of this embodiment further includes a passive component 160, in which the passive component 160 is disposed in the redistribution layer RDL, and is electrically connected to the redistribution layer RDL. In this embodiment, the fabrication of the passive component 160 may be integrated with the fabrication of the redistribution layer RDL.

To sum up, some embodiments of the disclosure provide a physiological sensing device including a coupling dielectric stacked layer disposed below the coupling sensing electrode. The coupling dielectric stacked layer may not only increase the coupling capacitance between the sensing electrode and the organism to be measured, but also improve the sensing sensitivity of the physiological sensing device. In addition, in the embodiments of the disclosure, the coupling dielectric stacked layer in the physiological sensing device has properties such as scratch resistance, abrasion resistance, and/or moisture resistance, so as to improve the reliability of the physiological sensing device to a certain extent. In addition, some embodiments of the disclosure provide a physiological sensing device, which includes a conductive layer that may be used to further improve the sensing sensitivity of the physiological sensing device.

Although the disclosure has been described with reference to the above embodiments, it will be apparent to one of ordinary skill in the art that modifications to the described embodiments may be made without departing from the spirit of the disclosure. Accordingly, the scope of the disclosure will be defined by the attached claims and their equivalents and not by the above detailed descriptions.

What is claimed is:

1. A physiological sensing device, suitable for sensing physiological signal of an organism, the physiological sensing device comprising:
a sensing chip,
a coupling sensing electrode, electrically connected to the sensing chip; and
a coupling dielectric stacked layer, covering the coupling sensing electrode and located between the coupling sensing electrode and the organism, wherein the coupling dielectric stacked layer comprises a first dielectric layer and a second dielectric layer, a dielectric constant of the second dielectric layer is greater than a dielectric constant of the first dielectric layer, and the second dielectric layer is located between the first dielectric layer and the organism.

2. The physiological sensing device according to claim 1, further comprising:
a fabric layer, wherein the second dielectric layer is located between the first dielectric layer and the fabric layer, and the fabric layer is located between the organism and the second dielectric layer.

3. The physiological sensing device according to claim 1, further comprising:
a stress compensation layer, disposed between the coupling sensing electrode and the coupling dielectric stacked layer.

4. The physiological sensing device according to claim 1, wherein the coupling dielectric stacked layer further comprises:
a third dielectric layer, disposed between the first dielectric layer and the coupling sensing electrode, wherein a dielectric constant of the third dielectric layer is greater than the dielectric layer of the first dielectric layer.

5. The physiological sensing device according to claim 4, wherein the first dielectric layer comprises a plurality of dielectric patterns separated from each other, the second dielectric layer comprises a plurality of dielectric patterns separated from each other, and the third dielectric layer comprises a plurality of dielectric patterns separated from each other.

6. The physiological sensing device according to claim 5, wherein the second dielectric layer is in contact with the third dielectric layer.

7. The physiological sensing device according to claim 5, wherein the first dielectric layer is filled between the dielectric patterns separated from each other of the third dielectric layer.

8. The physiological sensing device according to claim 4, wherein the second dielectric layer comprises a plurality of dielectric patterns separated from each other, and the third dielectric layer comprises a plurality of dielectric patterns separated from each other.

9. The physiological sensing device according to claim 8, wherein the second dielectric layer is separated from the third dielectric layer by the first dielectric layer.

10. The physiological sensing device according to claim 8, wherein the first dielectric layer is filled between the dielectric patterns separated from each other of the third dielectric layer.

11. The physiological sensing device according to claim 1, wherein the second dielectric layer comprises a plurality of dielectric patterns separated from each other.

12. The physiological sensing device according to claim 1, further comprising:
a redistribution layer comprising the coupling sensing electrode; and
a passive component, disposed in the redistribution layer and electrically connected to the redistribution layer.

13. The physiological sensing device according to claim 1, wherein the dielectric constant of the second dielectric layer is between 7 and 10000, and the dielectric constant of the first dielectric layer is between 3 and 7.

14. The physiological sensing device according to claim 1, wherein the second dielectric layer is embedded in the first dielectric layer, and an outer surface of the first dielectric layer is coplanar with an outer surface of the second dielectric layer.

15. A physiological sensing device, suitable for sensing physiological signal of an organism, the physiological sensing device comprising:
a sensing chip,
a coupling sensing electrode, electrically connected to the sensing chip;
a coupling dielectric stacked layer, covering the coupling sensing electrode and located between the coupling sensing electrode and the organism, wherein the coupling dielectric stacked layer comprises a stress compensation layer and a first dielectric layer, and the first dielectric layer is located between the stress compensation layer and the organism; and
a second dielectric layer, disposed between the stress compensation layer and the coupling sensing electrode.

16. A physiological sensing device, suitable for sensing physiological signal of an organism, the physiological sensing device comprising:
a sensing chip,
a coupling sensing electrode, electrically connected to the sensing chip;
a coupling dielectric layer, covering the coupling sensing electrode and located between the coupling sensing electrode and the organism; and
a conductive layer, disposed on the coupling dielectric layer, wherein the conductive layer and the coupling sensing electrode are respectively located on two sides of the coupling dielectric layer.

17. The physiological sensing device according to claim 16, wherein the conductive layer is electrically floating.

18. The physiological sensing device according to claim 16, wherein the conductive layer and the coupling sensing electrode are separated from each other by the coupling dielectric layer.

19. The physiological sensing device according to claim 16, wherein the coupling dielectric layer comprises a single-layer dielectric layer or a multi-layer dielectric layer stacked on top of each other.

20. The physiological sensing device according to claim 16, wherein a first capacitor is formed by the organism and the conductive layer, a second capacitor is formed by the conductive layer and the coupling sensing electrode, and the first capacitor and the second capacitor are connected in parallel.
